SEQUENCE LISTING (15 sequences)

SEQ ID NO: 1 - DNA, 1546 bp, Artificial Sequence
  Synthesized
atggactacaaagacgatgacgacaaggaagctgcaccctgtgactgtgatggaggtacacaaaaagaaaaaggcccatattatacacaccttggggcaggaccaagtgtggctgctgtcagggagctcatggagactaggtttggccagaaggggaaggcaatccggattgagaagatagtgttcacggggaaggaagggaagagctctcagggctgcccggtcgccaagtgggtgatcagaagaagtggtcctgaagagaagcttatttgtttggttcgtgagcgtgtagaccatcactgttcgacggctgtgatagttgtccttatcctgctgtgggaaggtatccctcgcctgatggctgaccgcctgtacaaagagctcactgagaacttgaggtcctacagcggacatcccacagaccgaagatgtaccctcaacaaaaagcgtacctgcacctgtcaaggcatcgacccaaaaacctgcggagcgtccttctcctttggctgttcgtggagcatgtatttcaacggctgtaagtttgggaggagtgaaaaccccagaaaattcagacttgctccaaactaccccttacataactactataagagaattactggaatgagttctgaaggaagtgacgtgaaaaccgggtggatcattccagaccgcaagaccctcataagcagagaggaaaaacagcttgaaaagaatttacaagaattggctacagtattagctccactttacaagcagatggctccagttgcttatcaaaatcaggtggaatatgaagaagttgctggagactgtcgacttggaaatgaagaggggcgtcctttctctggtgtcacctgttgcatggatttttgtgcccattctcacaaggacattcacaacatgcacaacggaagcaccgtggtgtgtacgttgattcgagcagatggccgtgacacaaattgtcccgaggatgaacaactccacgtcctgccactataccggcttgcagacactgatgaatttggctccgtggaagggatgaaggccaaaatcaaatctggggccatccaagtcaatgggccaaccaggaagaggcgactacgttttactgagcctgttcctcgatgtgggaagagggccaaaatgaagcagaaccacaataaatcaggttcacacaacactaagagcttttcatcagcctcatctacttctcacctagtgaaagacgaatctacagacttctgtcccctgcaggcttcctccgcagaaacatctacctgtacgtacagtaaaacagcctcaggtgggtttgcagaaacaagtagtattctccactgcacaatgccttctggagcacacagtggtgctaatgcagctgctggggaatgtactggaacggtgcagcctgccgaggtggctgctcatcctcaccagtctcttcccacagccgattctcccgttcatgctgagcctctcactagtccatctgagcagctaacttctaaccagtcaaaccagcagctccctctcctcagcaattctcaga

SEQ ID NO: 2 - DNA, 108 bp, Artificial Sequence
  Synthesized
atggactacaaagacgatgacgacaaggaagctgcaccctgtgactgtgatggaggtacacaaaaagaaaaaggcccatattatacacaccttggggcaggaccaagt

SEQ ID NO: 3 - DNA, 137 bp, Artificial Sequence
  Synthesized
tgatcacccacttggcgaccgggcagccctgagagctcttcccttccttccccgtgaacactggattgccttccccttctggccaaacctagtctccatgagctccctgacagcagccacacttggtcctgccccaa

SEQ ID NO: 4 - DNA, 150 bp, Artificial Sequence
  Synthesized
tcgccaagtgggtgatcagaagaagtggtcctgaagagaagcttatttgtttggttcgtgagcgtgtagaccatcactgttcgacggctgtgatagttgtccttatcctgctgtgggaaggtatccctcgcctgatggctgaccgcctgt

SEQ ID NO: 5 - DNA, 150 bp, Artificial Sequence
  Synthesized
ccaaaggagaaggacgctccgcaggtttttgggtcgatgccttgacaggtgcaggtacgctttttgttgagggtacatcttcggtctgtgggatgtccgctgtaggacctcaagttctcagtgagctctttgtacaggcggtcagccatc

SEQ ID NO: 6 - DNA, 150 bp, Artificial Sequence
  Synthesized
gagcgtccttctcctttggctgttcgtggagcatgtatttcaacggctgtaagtttgggaggagtgaaaaccccagaaaattcagacttgctccaaactaccccttacataactactataagagaattactggaatgagttctgaaggaa

SEQ ID NO: 7 - DNA, 150 bp, Artificial Sequence
  Synthesized
ggagccatctgcttgtaaagtggagctaatactgtagccaattcttgtaaattcttttcaagctgtttttcctctctgcttatgagggtcttgcggtctggaatgatccacccggttttcacgtcacttccttcagaactcattccagta

SEQ ID NO: 8 - DNA, 150 bp, Artificial Sequence
  Synthesized
actttacaagcagatggctccagttgcttatcaaaatcaggtggaatatgaagaagttgctggagactgtcgacttggaaatgaagaggggcgtcctttctctggtgtcacctgttgcatggatttttgtgcccattctcacaaggacat

SEQ ID NO: 9 - DNA, 149 bp, Artificial Sequence
  Synthesized
cccattctcacaaggacattcacaacatgcacaacggaagcaccgtggtgtgtacgttgattcgagcagatggccgtgacacaaattgtcccgaggatgaacaactccacgtcctgccactataccggcttgcagacactgatgaattt

SEQ ID NO: 10 - DNA, 150 bp, Artificial Sequence
  Synthesized
gcttgcagacactgatgaatttggctccgtggaagggatgaaggccaaaatcaaatctggggccatccaagtcaatgggccaaccaggaagaggcgactacgttttactgagcctgttcctcgatgtgggaagagggccaaaatgaagca

SEQ ID NO: 11 - DNA, 150 bp, Artificial Sequence
  Synthesized
acaggtagatgtttctgcggaggaagcctgcaggggacagaagtctgtagattcgtctttcactaggtgagaagtagatgaggctgatgaaaagctcttagtgttgtgtgaacctgatttattgtggttctgcttcattttggccctctt

SEQ ID NO: 12 - DNA, 150 bp, Artificial Sequence
  Synthesized
ccgcagaaacatctacctgtacgtacagtaaaacagcctcaggtgggtttgcagaaacaagtagtattctccactgcacaatgccttctggagcacacagtggtgctaatgcagctgctggggaatgtactggaacggtgcagcctgccg

SEQ ID NO: 13 - DNA, 150 bp, Artificial Sequence
  Synthesized
tctgagaattgctgaggagagggagctgctggtttgactggttagaagttagctgctcagatggactagtgagaggctcagcatgaacgggagaatcggctgtgggaagagactggtgaggatgagcagccacctcggcaggctgcaccg

SEQ ID NO: 14 - DNA, 22 bp, Artificial Sequence
  Synthesized
atggactacaaagacgatgacg

SEQ ID NO: 15 - DNA, 22 bp, Artificial Sequence
  Synthesized
tctgagaattgctgaggagagg

(19) United States
(12) Patent Application Publication
(10) Pub. No.: US 2023/0407359 A1
(43) Pub. Date: Dec. 21, 2023

(54) A HIGH-THROUGHPUT AUTOMATED GENE SYNTHESIS DEVICE BASED ON CLUSTER ARRAY

(71) Applicant: DYNEGE BIOSCIENCE CORPORATION, Shanghai (CN)

(72) Inventors: Mancang ZHANG, Shanghai (CN); Cailing HOU, Shanghai (CN); Dongping YU, Shanghai (CN)

(21) Appl. No.: 18/251,803

(22) PCT Filed: May 18, 2021
(86) PCT No.: PCT/CN2021/094310

§ 371 Date: May 4, 2023

(30) Foreign Application Priority Data
Mar. 29, 2021 (CN) 202110344493.0

(51) Int. Cl. C12P 19/34; B01J 19/00; C12N 9/12

(52) U.S. Cl. CPC C12P 19/34; B01J 19/0046; C12N 9/1276; B01J 2219/00313; B01J 2219/00722

(57) ABSTRACT

A high-throughput automated gene synthesis device based on a cluster array includes a substrate and a microwell plate; the substrate is provided with a plurality of clusters of micropores; the inner wall surface of the micropores is chemically modified as a solid phase carrier for nucleic acid synthesis, or the micropore is filled with solid phase carriers; the clusters of micropores are arranged in a cluster array and each cluster of micropores has the same size and corresponding position as each well on the microwell plate. When using the device to synthesize oligonucleotides, by automatically recovering the synthesized oligonucleotides into a standard SBS plate of the corresponding size under the device, the oligonucleotide pool for each gene is formed. The yield of oligonucleotides is in picomole level, which is used for subsequent polymerase-mediated gene assembly (PCA) or ligase-mediated gene assembly (LCR) without amplification.

### TECHNICAL FIELD

The present invention relates to synthetic biology and microelectromechanical systems (MEMS), particularly to a high-throughput automated gene synthesis device using cluster arrays.

### BACKGROUND OF THE INVENTION

Oligonucleotide synthesis and gene assembly, as powerful tools of synthetic biology, are widely used in molecular biology (including library construction, sequencing, gene editing, etc.), protein engineering, metabolic engineering, biomedical engineering and genetic testing and other fields.

In the traditional commercial solid-phase oligonucleotide synthesis method, each oligonucleotide is synthesized in a separate synthesis tube or in a well in a synthesis plate, and the yield of each nucleic acid is high, which is usually in nanomole level. However, such synthesis method consumes large amounts of reagents and thus the cost is high. Besides, during gene synthesis, mixing oligonucleotides into an oligonucleotide pool is an essential step for performing subsequent gene assembly. Poor quality of manual mixed oligonucleotides pools may cause the pooling problem in subsequent gene assembly process. The microarray-based high-throughput synthesis method has been widely studied, which has the advantages of high throughput (up to millions of different oligonucleotide sequences can be synthesized on a single chip) and low cost. However, the yield of single oligonucleotide synthesis is relatively low, generally in the femtomole level (generally from 10⁵ to 10¹² molecules/sequence, even not enough to trigger a PCR reaction). Therefore, multiple PCR amplifications of oligonucleotide pool are required prior to subsequent gene assembly. When all the synthesized nucleic acid sequences on a chip need to be cut down into a mixture, to avoid interactions between different sequences in the mixture, the sequence and the amount of each nucleic acid need to be carefully designed. And during gene synthesis, the oligonucleotide mixture needs to be divided into several oligonucleotide sub-pools using universal primers and other methods, and then further gene assembly is performed. The mixing operation is complex which may cause the depooling problem. The nucleic acid synthesis method based on microfluidic device has been reported that it has the benefits of no cross-contamination, saving reagents, and a high synthesis yield (100 pmol level, which can directly apply to gene assembly without amplification). However, the microfluidic device itself needs to introduce a micropump and microvalve and the like, which makes its structure relatively complex, operation troublesome and efficiency reduced. Therefore, microfluidic-device-based synthesis approach has not been widely applied and commercialized. In addition, some companies have reported a commercialized DNA synthesis platform based on semiconductor silicon chips. This unique honeycomb microwell design reduces the reaction volume by one million times. But this method requires relatively complex substrate processing technology, unique liquid processing technology and substrate fixing device, resulting in a high cost of synthesizing nucleic acid. Although no manual mixing or splitting process is required during semiconductor silicon chips-based synthesis, the yield is still not high enough to perform following gene assembly without PCR amplification. Therefore, it is necessary to continue to develop nucleic acid synthesis technologies that can produce suitable amounts of synthetic oligonucleotides and has the advantages of simplicity, low cost, high-throughput and potential of commercializing automated gene assembly.

### SUMMARY OF THE INVENTION

The purpose of the present invention is to provide a high-throughput gene synthesis device based on cluster arrays, which can synthesize oligonucleotides of various lengths on the same plate. This method is more conducive to the PCA splicing of oligonucleotides or ligase-mediated assembly using a combination of short primers and long oligonucleotides.

The high-throughput gene synthesis device based on cluster arrays provided by the present invention includes a substrate and a microwell plate;
the substrate is provided with a plurality of clusters of micropores; the inner wall surface of the micropore is chemically modified as a solid phase carrier for nucleic acid synthesis, or the micropore is filled with solid phase carriers;
a plurality of clusters of the micropores are arranged in a cluster array, and each cluster of the micropores has the same size and corresponding position as each well on the microwell plate.

In the gene synthesis device mentioned above, the micropore is a funnel-shaped micropore or a cylindrical micropore;
the opening of the funnel-shaped micropore is a large opening end.

In the gene synthesis device mentioned above, the substrate can be a silicon wafer, and the micropore can be prepared by the MEMS micro-nano processing method.

In the above-mentioned gene synthesis device, the substrate can be a polymer plastic plate, and the micropore can be prepared by 3D printing or injection molding.

In the above-mentioned gene synthesis device, the solid phase carriers can be glass microspheres or polystyrene microspheres;
the solid phase carriers are immobilized in the micropore as follows:
mixing the solid phase carriers with high-density polyethylene microspheres, and sintering;
the surface of the solid phase carrier and the surface of the inner nanopore are modified with bonding arms as the starting point of the oligonucleotide synthesis.

In the above-mentioned gene synthesis device, each cluster of the micropores includes 4 to 68 micropores;
the microwell plate is a standard SBS plate, such as a 96-well plate, a 384-well plate or a 1536-well plate. Correspondingly, 96 clusters of micropores, 384 clusters of micropores, or 1536 clusters of micropores are arranged on the substrate;
each cluster of micropores on the substrate is arranged corresponding to each well of the microwell plate, so as to facilitate subsequent automated gene splicing and synthesis: each cluster of the cluster array corresponds to one well of the lower SBS standard microwell plate and the oligonucleotides synthesized in all pores in each cluster can meet the requirement of full-length splicing of a gene.

When using the gene synthesis device of the present invention to synthesize oligonucleotides, the following steps can be performed:
adding phosphoramidite monomers or auxiliary reagents to the micropores in the gene synthesis device by using a liquid dispensing device, and reacting on the solid phase carriers to obtain oligonucleotides;
matching the substrate with the microwell plate, and recovering the oligonucleotides obtained in each cluster of the micropores into one general well in the microwell plate;
The mentioned liquid separation device is a micro-nano liquid dispenser head. It differs from existing industrial inkjet printing nozzles in that it can perform multi-channel fluid distribution of nanoliter and microliter volume upgrades, which is a magnitude larger in fluid volume compared to the pico-liter upgrade of inkjet printing nozzles, making it more suitable for liquid separation during oligonucleotide synthesis for gene synthesis purposes. During the synthesis process, positive pressure and/or negative pressure are applied to gradually pass various auxiliary chemical solutions required for synthesis through the surface of the solid phase carriers. After the synthesis is completed, ammonolysis is performed and the length of synthetic oligonucleotide varies from 15 to 350 bases.

The device of the present invention can synthesize oligonucleotides of various lengths on the same plate and is more conducive to the polymerase-mediated gene assembly splicing of oligonucleotides or ligase-mediated assembly using a combination of short primers and long oligonucleotides. Compared with the traditional multi-step splicing method using short oligonucleotides, the one-step splicing method can be adopted to accomplish gene assembly using ultra-long oligonucleotides as initial splicing elements, which is easier to automate.

When synthesizing oligonucleotides using the device of the present invention, the synthesized oligonucleotides are automatically recovered into the corresponding size of standard SBS plates (96-well plate, 384-well plate, 1536-well plate, etc.) under the device to form an oligonucleotide pool for each gene. The yield of oligonucleotides is at the picomole level, which can meet the needs of subsequent gene assembly through polymerase chain reaction (PCR) or ligation chain reaction (LCR) without amplification. After error correction, the oligonucleotides can be used to complete the full-length assembly of genes, realizing high-throughput automated gene synthesis.

### DESCRIPTION OF THE DRAWINGS

FIG. 1 shows a process flow of processing funnel-shaped micropores arranged in clusters on a silicon wafer.

FIG. 2 shows a SEM image of a funnel-shaped micropore on a silicon wafer (FIG. 2A) and an image of loaded microspheres in a micropore (FIG. 2B).

FIG. 3 shows a nucleic acid synthesis device based on funnel-shaped micropores arranged in clusters on a silicon wafer (when a standard 96-well plate is used for recovering).

FIG. 4 shows a nucleic acid synthesis device based on funnel-shaped micropores arranged in clusters on a silicon wafer (when a standard 384-well plate is used for recovering).

FIG. 5 shows a nucleic acid synthesis device based on funnel-shaped micropores arranged in clusters on a silicon wafer (when a standard 1536-well plate is used for recovering).

FIG. 6 shows a nucleic acid synthesis device based on funnel-shaped micropores arranged in cluster arrays on a polymer plastic plate (when a standard 96-well plate is used for recovering).

FIG. 7 shows a nucleic acid synthesis device based on funnel-shaped micropores arranged in cluster arrays on a polymer plastic plate (when a standard 384-well plate is used for recovering).

FIG. 8 shows a dispensing device based on a micro-nano dispensing head.

FIG. 9 shows a flow chart of the overall process of gene synthesis.

FIG. 10 is a graph showing the detection by capillary electrophoresis (150 nt) after oligonucleotide synthesis.

FIG. 11 is a graph showing the detection of PCA products by capillary electrophoresis.

FIG. 12 is a graph showing the detection of PCR products by capillary electrophoresis.

FIG. 13 is a graph showing the detection of error-corrected products by capillary electrophoresis.

FIG. 14 shows the sequencing results of strain 1 (including FIG. 14-A, FIG. 14-B and FIG. 14-C).

FIG. 15 shows the sequencing results of strain 2 (including FIG. 15-A, FIG. 15-B and FIG. 15-C).

### DETAILED DESCRIPTION OF THE INVENTION

The experimental methods in the following examples are conventional methods unless otherwise specified.

The materials, reagents, and etcetera used in the following examples can be obtained from commercial sources unless otherwise specified.

### Example 1: Preparation of Funnel-Shaped Micropores Arranged in Clusters on Silicon Wafers and Loading of Microspheres

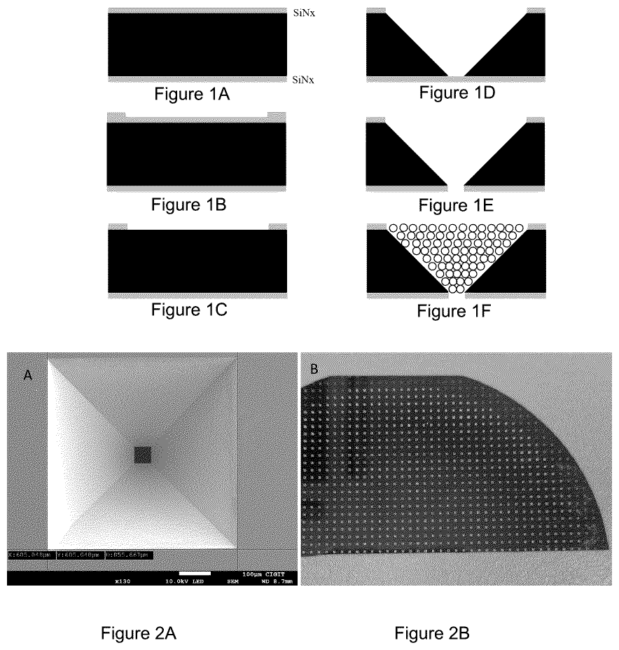

The process flow of processing funnel-shaped micropores arranged in clusters on a silicon wafer is shown in FIG. 1:
(1) On both sides of an 8-inch silicon wafer with a thickness of 400 jam and double-sided polished, a 20 nm thick layer of silicon nitride is deposited by chemical vapor deposition, as shown in FIG. 1A.
(2) Photoresist is coated on the front side of the silicon wafer, and the mask plate is used for photolithography, as shown in FIG. 1B.
(3) The silicon nitride layer was removed by reactive ion etching, as shown in FIG. 1C.
(4) The silicon is etched to the bottom silicon nitride layer using a wet etching solution of potassium hydroxide with an etching angle of 54.7°, as shown in FIG. 1D.
(5) The silicon nitride layer at the small opening of the inverted wedge-shaped hole on the back of the silicon wafer is removed by ultrasonic (or reactive ion etching), creating a through hole, as shown in FIG. 1E.

FIG. 2A illustrates a scanning electron microscope (SEM) image with an opening side length of 605 jam above a funnel-shaped micropore.
(6) The funnel-shaped microhole obtained by the above process is a reaction cavity for oligonucleotides. Polystyrene microspheres or glass microspheres (solid phase carriers) are loaded into the funnel-shaped micropores. The microspheres are mixed with high-density polyethylene spheres in a certain ratio (such as 1:1) and sintered at 140° C. for 45 minutes. The high-density polyethylene spheres are used to achieve physical bonding between the solid phase carriers and the inner wall of the funnel-shaped micropores, thus fixing the solid phase carriers inside the through hole, as shown in FIG. 1F. The image after loading the solid phase carriers into the funnel-shaped micropores is shown in FIG. 2B. Subsequent oligonucleotide chemical synthesis reactions are carried out on the solid phase carriers, and the carrier surface is connected with intermediates, which can serve as the starting point for nucleic acid synthesis.

### Example 2: Nucleic Acid Synthesis Device Based on Funnel-Shaped Micropores Arranged in Clusters on a Silicon Wafer

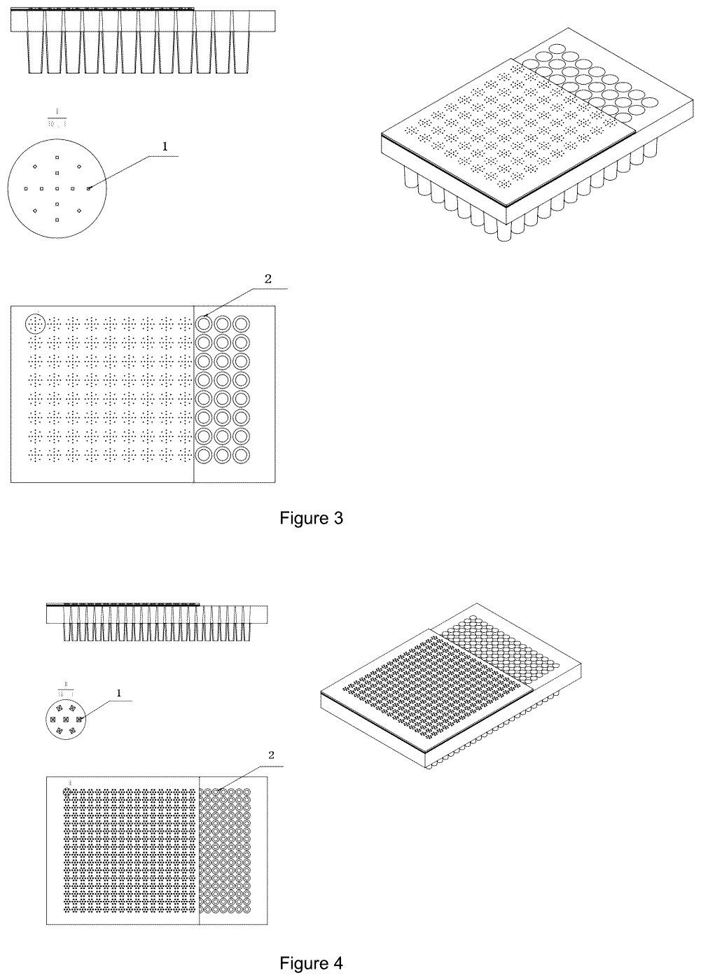

FIGS. 3, 4 and 5 show three different throughputs of nucleic acid synthesis devices for the purpose of gene synthesis.

As shown in FIG. 3, there are 13 funnel-shaped micropores 1 (oligonucleotide synthesis pores) on a silicon wafer form a cluster. The silicon wafer is combined with a 96-wellplate below. A dispensing system based on micro-nano dispensing heads is used to conduct the chemical synthesis of oligonucleotides on the solid-phase carriers in the funnel-shaped micropore 1, and the oligonucleotides synthesized in the 13 funnel-shaped micropores 1 are automatically recovered into a large well 2 (gene splicing well), followed by automated one-step splicing to synthesize a gene.

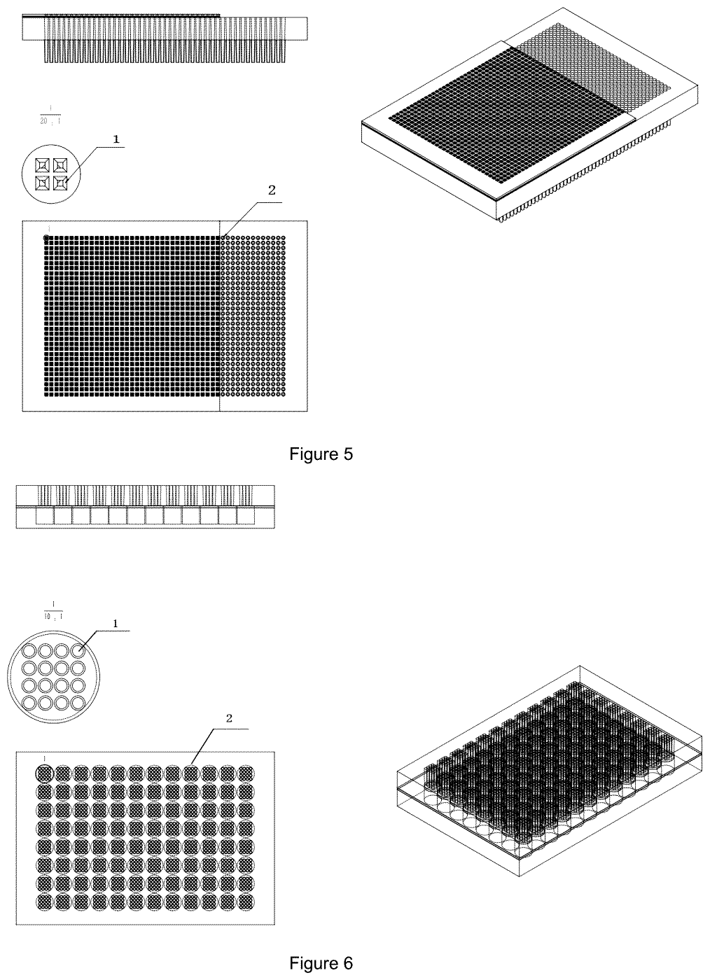

FIGS. 4 and 5 are schematic diagrams of the devices for synthesizing 384 and 1536 genes, with 7 and 4 funnel-shaped micropores 1 as a cluster, respectively. The arrangement and density of each cluster of funnel-shaped micropores can be adjusted according to the length of the gene to be synthesized, the crystal orientation and thickness of the silicon wafer, the size of the upper and lower openings of the funnel-shaped micropores, and the like.

### Example 3: Nucleic Acid Synthesis Device Based on Funnel-Shaped Micropores Arranged in Cluster Arrays on a Polymer Plastic Plate

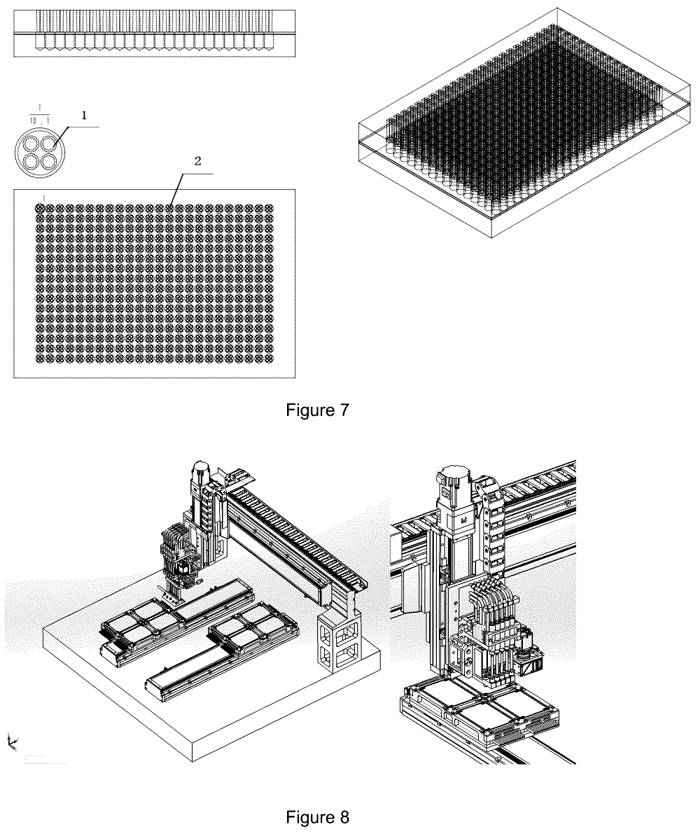

FIGS. 6 and 7 illustrate two different throughputs of nucleic acid synthesis devices for the purpose of gene synthesis.

As shown in FIG. 6, there are 1536 funnel-shaped micropores 1 (oligonucleotide synthesis pores) with large upper opening and small lower opening arranged on the polymer plastic plate. The funnel-shaped micropore 1 is loaded with fits obtained by sintering solid phase carriers and high-density polyethylene spheres. A dispensing system based on micro-nano dispensing heads is used to conduct the synthesis of oligonucleotides on the solid phase carriers in the micropore. 16 pores form a cluster, and 16 kinds of oligonucleotides are synthesized in the pore. The oligonucleotides are automatically recovered into one large well 2 (gene splicing well) of the 96-well plate, i.e., the assembly of one gene can be completed in one large well 2, and the assembly of 96 genes can be completed simultaneously.

As shown in FIG. 7, 4 pores form a cluster and 384 genes can be assembled simultaneously.

### Example 4: Oligonucleotide Synthesis

Nucleic acid synthesis reactions were carried out on the solid phase carriers in funnel-shaped micropores on silicon wafers or in micropores in polymer plastic plates: standard chemical synthesis methods (including the steps of deprotection, coupling, capping and oxidation) may be used.

Specific chemical synthesis implementation method is as follows: using different micro-nano dispensing heads for four or more different (deoxygenated/modified) nucleotide monomer solutions and activators and/or auxiliary reagents, according to the sequence information to be synthesized in each synthesis pore. The liquid type, position and liquid amount of the dispensing head were automatically controlled to complete the chemical synthesis of nucleic acid.

The liquid dispensing device used in the synthesis process is a micro-nanoliquid dispensing head, as shown in FIG. 8. The overall frame adopts a high-precision marble platform as the installation reference surface, a gantry-type 4-axis servo positioning transmission, and the X-axis, Y1-axis, and Y2-axis choose a high-precision linear motor drive, with Z-axis precision screw drive module. The core dispensing element is a micro-nano dispensing head with high-speed response accuracy. The liquid dispensing process is assisted by the visual positioning of the workstation; the waste liquid generated during the synthesis process is collected into a waste liquid bottle by negative pressure generated by a vacuum pump. Among them, two identical parallel stations, Y1 and Y2, are set in the Y direction, and the two axes alternately dispense the liquid to improve the synthesis throughput and efficiency; 4 of the above cluster array plates can be installed on the Y1/Y2 axis fixture, and the array plate is driven to the zero point in the Y axis direction. Five micro-nano dispensing heads and auxiliary positioning cameras are installed on the Z-axis. After the X-axis drives the Z-axis camera to locate the synthetic dispensing origin, the X-axis is continuously positioned in the position control mode to drive the micro-nano liquid dispensing head to selectively dispense into the micropores corresponding to the first row, and the X-axis returns to zero point. Next, the Y-axis drives the fixture to advance one row, and the X-axis flight mode is used to selectively dispense liquid to the second row, and so on, to complete the automated synthesis of 1 base of 4 cluster array plates.

A purified 150 nt oligonucleotide product was detected on the 2100 Bioanalyzer using the capillary electrophoresis kit, RNA Pico 6000 Kit (Agilent, Cat. No. 5067-1513).

### Example 5. Gene Synthesis

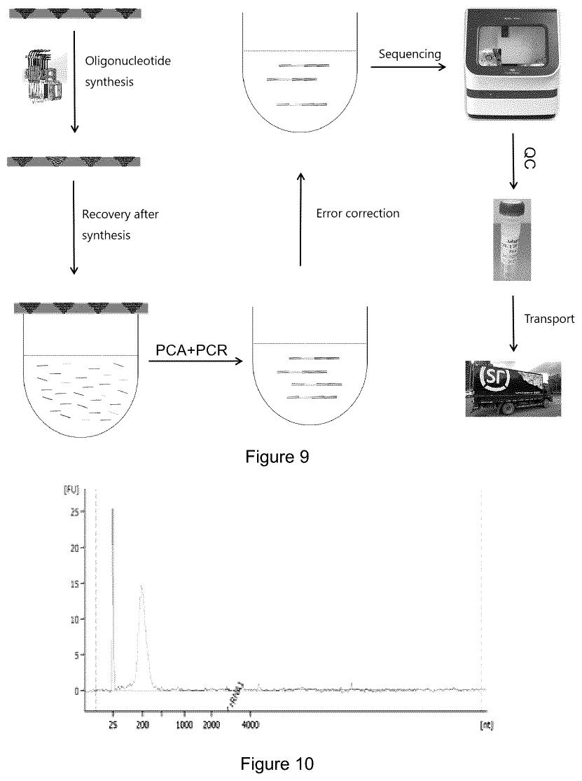

The flow chart of the overall process of gene synthesis is shown in FIG. 9.

The oligonucleotides synthesized in each cluster of funnel-shaped micropores are recovered into one well of the corresponding multi-well plate (96 wells, 384 wells, 1536 wells), and gene splicing and assembly are performed directly in the corresponding wells to achieve automated parallel synthesis of 96 or 384 or 1536 genes.

For example, to synthesize the 1546-base CDS sequence (with a 27-base tag sequence at the N-terminus for protein purification) of the methylcytosine dioxygenase (Tet1, mouse) gene, the sequence is as follows (SEQ ID NO: 1 in the Sequence Listing):

5′-ATGGACTACAAAGACGATGACGACAAGGAAGCTGCACCCTGTGACTG
TGATGGAGGTACACAAAAAGAAAAAGGCCCATATTATACACACCTTGGGG
CAGGACCAAGTGTGGCTGCTGTCAGGGAGCTCATGGAGACTAGGTTTGGC
CAGAAGGGGAAGGCAATCCGGATTGAGAAGATAGTGTTCACGGGGAAGGA
AGGGAAGAGCTCTCAGGGCTGCCCGGTCGCCAAGTGGGTGATCAGAAGAA
GTGGTCCTGAAGAGAAGCTTATTTGTTTGGTTCGTGAGCGTGTAGACCAT
CACTGTTCGACGGCTGTGATAGTTGTCCTTATCCTGCTGTGGGAAGGTAT
CCCTCGCCTGATGGCTGACCGCCTGTACAAAGAGCTCACTGAGAACTTGA
GGTCCTACAGCGGACATCCCACAGACCGAAGATGTACCCTCAACAAAAAG
CGTACCTGCACCTGTCAAGGCATCGACCCAAAAACCTGCGGAGCGTCCTT
CTCCTTTGGCTGTTCGTGGAGCATGTATTTCAACGGCTGTAAGTTTGGGA
GGAGTGAAAACCCCAGAAAATTCAGACTTGCTCCAAACTACCCCTTACAT
AACTACTATAAGAGAATTACTGGAATGAGTTCTGAAGGAAGTGACGTGAA
AACCGGGTGGATCATTCCAGACCGCAAGACCCTCATAAGCAGAGAGGAAA
AACAGCTTGAAAAGAATTTACAAGAATTGGCTACAGTATTAGCTCCACTT
TACAAGCAGATGGCTCCAGTTGCTTATCAAAATCAGGTGGAATATGAAGA
AGTTGCTGGAGACTGTCGACTTGGAAATGAAGAGGGGCGTCCTTTCTCTG
GTGTCACCTGTTGCATGGATTTTTGTGCCCATTCTCACAAGGACATTCAC
AACATGCACAACGGAAGCACCGTGGTGTGTACGTTGATTCGAGCAGATGG
CCGTGACACAAATTGTCCCGAGGATGAACAACTCCACGTCCTGCCACTAT
ACCGGCTTGCAGACACTGATGAATTTGGCTCCGTGGAAGGGATGAAGGCC
AAAATCAAATCTGGGGCCATCCAAGTCAATGGGCCAACCAGGAAGAGGCG
ACTACGTTTTACTGAGCCTGTTCCTCGATGTGGGAAGAGGGCCAAAATGA
AGCAGAACCACAATAAATCAGGTTCACACAACACTAAGAGCTTTTCATCA
GCCTCATCTACTTCTCACCTAGTGAAAGACGAATCTACAGACTTCTGTCC
CCTGCAGGCTTCCTCCGCAGAAACATCTACCTGTACGTACAGTAAAACAG
CCTCAGGTGGGTTTGCAGAAACAAGTAGTATTCTCCACTGCACAATGCCT
TCTGGAGCACACAGTGGTGCTAATGCAGCTGCTGGGGAATGTACTGGAAC
GGTGCAGCCTGCCGAGGTGGCTGCTCATCCTCACCAGTCTCTTCCCACAG
CCGATTCTCCCGTTCATGCTGAGCCTCTCACTAGTCCATCTGAGCAGCTA
ACTTCTAACCAGTCAAACCAGCAGCTCCCTCTCCTCAGCAATTCTCAGA-
3′

The process of gene synthesis is as follows:

(1) Design of Oligonucleotide Sequences According to the DNA Sequence of the Target Gene

By using DNAWorks, the target DNA sequence was codon-optimized and split into 12 sequence fragments connected end to end. And each segment was about 150 nt in length, the average number of bases in the overlapping region was about 20 bp, and the Tm value was 62° C. The head and tail primers for amplifying the 1546 nt fragment, Pa and Pb, were designed. The sequences of the 12 fragments and the head and tail primers are shown in Table 1:

TABLE 1
Seqs 1-12 and the sequences of the head and tail primers

| Name | Sequence (5′-3′) | Length (nt) |
|---|---|---|
| Seq1 | ATGGACTACAAAGACGATGACGACAAGGAAGCTGCACCCTGTGACTGTGATGGAGGTACACAA | 108 (corresponding to |
| | AAAGAAAAAGGCCCATATTATACACACCTTGGGGCAGGACCAAGT | positions 1-108 in above |
| | | CDS sequence) |
| Seq2 | TGATCACCCACTTGGCGACCGGGCAGCCCTGAGAGCTCTTCCCTTCCTTCCCCGTGAACACTGG | 150 (corresponding to |
| | ATTGCCTTCCCCTTCTGGCCAAACCTAGTCTCCATGAGCTCCCTGACAGCAGCCACACTTGGTC | positions 92-241 in above |
| | CTGCCCCAA | CDS sequence) |
| Seq3 | TCGCCAAGTGGGTGATCAGAAGAAGTGGTCCTGAAGAGAAGCTTATTTGTTTGGTTCGTGAGC | 150 (corresponding to |
| | GTGTAGACCATCACTGTTCGACGGCTGTGATAGTTGTCCTTATCCTGCTGTGGGAAGGTATCCCT | positions 224-373 in above |
| | CGCCTGATGGCTGACCGCCTGT | CDS sequence) |
| Seq4 | CCAAAGGAGAAGGACGCTCCGCAGGTTTTTGGGTCGATGCCTTGACAGGTGCAGGTACGCTTT | 150 (corresponding to |
| | TTGTTGAGGGTACATCTTCGGTCTGTGGGATGTCCGCTGTAGGACCTCAAGTTCTCAGTGAGCT | positions 357-506 in above |
| | CTTTGTACAGGCGGTCAGCCATC | CDS sequence) |
| Seq5 | GAGCGTCCTTCTCCTTTGGCTGTTCGTGGAGCATGTATTTCAACGGCTGTAAGTTTGGGAGGAG | 150 (corresponding to |
| | TGAAAACCCCAGAAAATTCAGACTTGCTCCAAACTACCCCTTACATAACTACTATAAGAGAATT | positions 488-637 in above |
| | ACTGGAATGAGTTCTGAAGGAA | CDS sequence) |
| Seq6 | GGAGCCATCTGCTTGTAAAGTGGAGCTAATACTGTAGCCAATTCTTGTAAATTCTTTTCAAGCTG | 150 (corresponding to |
| | TTTTTCCTCTCTGCTTATGAGGGTCTTGCGGTCTGGAATGATCCACCCGGTTTTCACGTCACTTC | positions 615-764 in above |
| | CTTCAGAACTCATTCCAGTA | CDS sequence) |
| Seq7 | ACTTTACAAGCAGATGGCTCCAGTTGCTTATCAAAATCAGGTGGAATATGAAGAAGTTGCTGGA | 150 (corresponding to |
| | GACTGTCGACTTGGAAATGAAGAGGGGCGTCCTTTCTCTGGTGTCACCTGTTGCATGGATTTTT | positions 744-893 in above |
| | GTGCCCATTCTCACAAGGACAT | CDS sequence) |
| Seq8 | CCCATTTCACAAGGACATTCACAACATGCACAACGGAAGCACCGTGGTGTGTACGTTGATTCG | 150 (corresponding to |
| | AGCAGATGGCCGTGACACAAATTGTCCCGAGGATGAACAACTCCACGTCCTGCCACTATACCG | positions 874-1023 in above |
| | GCTTGCAGACACTGATGAATTT | CDS sequence) |
| Seq9 | GCTTGCAGACACTGATGAATTTGGCTCCGTGGAAGGGATGAAGGCCAAAATCAAATCTGGGGC | 150 (corresponding to |
| | CATCCAAGTCAATGGGCCAACCAGGAAGAGGCGACTACGTTTTACTGAGCCTGTTCCTCGATGT | positions 1002-1151 in above |
| | GGGAAGAGGGCCAAAATGAAGCA | CDS sequence) |
| Seq10 | ACAGGTAGATGTTTCTGCGGAGGAAGCCTGCAGGGGACAGAAGTCTGTAGATTCGTCTTTCAC | 150 (corresponding to |
| | TAGGTGAGAAGTAGATGAGGCTGATGAAAAGCTCTTAGTGTTGTGTGAACCTGATTTATTGTGG | positions 1132-1281 in above |
| | TTCTGCTTCATTTTGGCCCTCTT | CDS sequence) |
| Seq11 | CCGCAGAAACATCTACCTGTACGTACAGTAAAACAGCCTCAGGTGGGTTTGCAGAAACAAGTA | 150 (corresponding to |
| | GTATTCTCCACTGCACAATGCCTTCTGGAGCACACAGTGGTGCTAATGCAGCTGCTGGGGAATG | positions 1262-1411 in above |
| | TACTGGAACGGTGCAGCCTGCCG | CDS sequence) |
| Seq12 | TCTGAGAATTGCTGAGGAGAGGGAGCTGCTGGTTTGACTGGTTAGAAGTTAGCTGCTCAGATG | 150 (corresponding to |
| | GACTAGTGAGAGGCTCAGCATGAACGGGAGAATCGGCTGTGGGAAGAGACTGGTGAGGATGA | positions 1397-1546 in above |
| | GCAGCCACCTCGGCAGGCTGCACCG | CDS sequence) |
| Pa | ATGGACTACAAAGACGATGACG | 22 |
| Pb | TCTGAGAATTGCTGAGGAGAGG | 22 |

(2) Synthesis of Oligonucleotides

The designed oligonucleotides were synthesized on the solid phase carriers in the funnel-shaped micropores in cluster arrays, and each cluster of oligonucleotides after ammonolysis was recovered into one well of the corresponding 96-well plate/384-well plate. The recovered oligonucleotide pools (Seq1-Seq12) were directly used for gene assembly without further purification steps. The oligonucleotide pools (Seq1-Seq12) were detected by capillary electrophoresis on Agilent 2100 Bioanalyzer, and the results are shown in FIG. 10.

(3) One-Step Gene Assembly Using the Polymerase Approach

The polymerase-based assembly method comprises two steps. The first step was Polymerase Cycling Assembly (PCA). 12 oligonucleotide fragments were used as primers and templates for each other to perform one-step splicing. PCR amplification of the spliced target fragments was carried out using the head and tail primers, Pa and Pb, and the product was tested by capillary electrophoresis on Agilent 2100 Bioanalyzer.

The PCA reaction system: 2×HiFi HotStart ReadyMix (Roche, Cat. No. KK2602), oligomix (4 pmoL each), and nuclease-free water to bring the volume to 4 μL (minimum reaction volume: 2 μL, maximum volume: 50 μL)

TABLE 2
PCA reaction system

| Components | Volume (μL) |
|---|---|
| 2 × HiFi HotStart Ready Mix | 2 |
| OligoMix (4 pmol) | 2 |
| Total | 4 |

The following reaction program was executed:

TABLE 3
PCA reaction program

| STEP | Temperature (° C.) | Time | |
|---|---|---|---|
| Intial Denaturation | 95 | 5 | min |
| 18 Cycles | 95 | 15 | s |
| | 60 | 15 | s |
| | 72 | 50 | s |
| Final Extension | 72 | 10 | min |

| | | |
|---|---|---|
| Hold | 12 | Hold |

The head and tail primers, Pa and Pb, were used to amplify the spliced target fragment by PCR. The reaction system:

TABLE 4
PCR reaction system

| Components | Volume (μL) |
|---|---|
| 2 × HiFi HotStart Ready Mix | 25 |
| Pa (10 uM) | 1 |
| Pb (10 uM) | 1 |
| PCA product | 1 |
| Nuclease-free water | 22 |
| Total | 50 |

The following PCR reaction program was executed:

TABLE 5
PCR reaction program

| STEP | Temperature (° C.) | Time | |
|---|---|---|---|
| Intial | 95 | 5 | min |
| Denaturation | | | |
| 18 Cycles | 95 | 15 | s |
| | 57 | 15 | s |
| | 72 | 50 | s |
| Final Extension | 72 | 10 | min |

| | | |
|---|---|---|
| Hold | 12 | Hold |

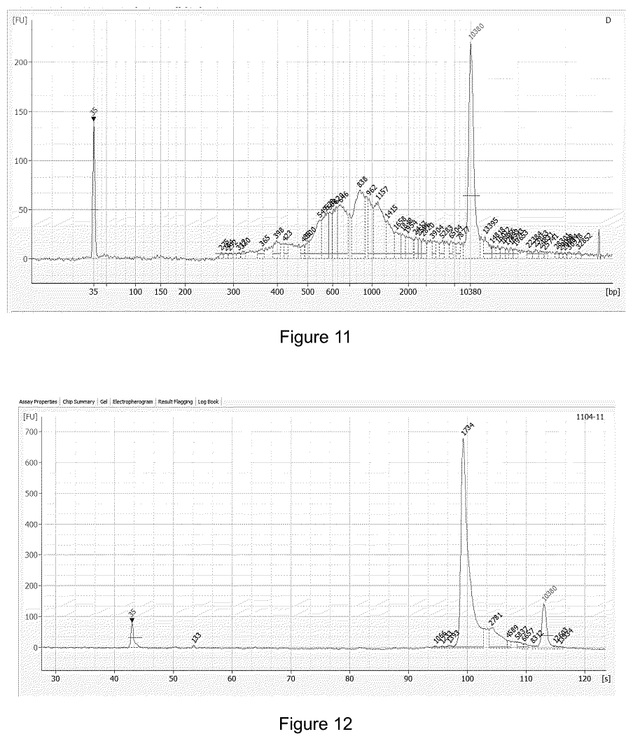

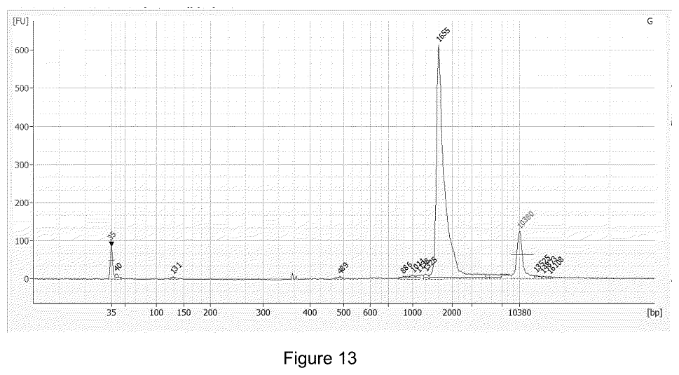

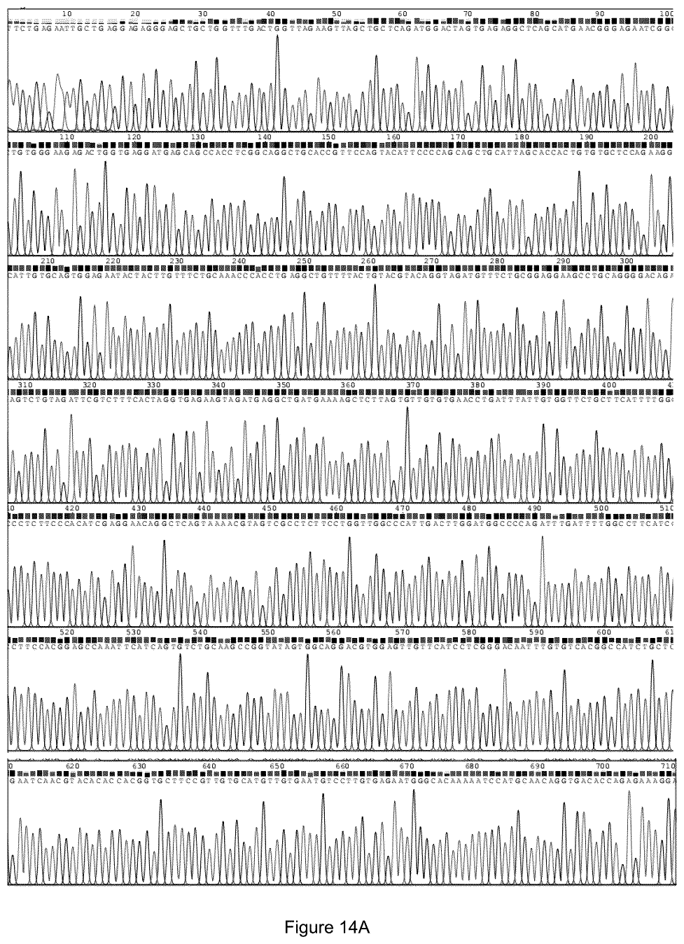

After the above PCA reaction, the Smear product after the fragment fusion was obtained, and then the PCA product was subjected to a PCR reaction to carry out the full-length fragment synthesis of the gene to obtain the 1546 bp target fragment using bilateral primers. CorrectASE enzyme (Thermo Fisher, Cat. No. A14972) was used for the error correction of the PCR product to obtain the final product for downstream cloning. The PCA, PCR, and error-corrected products were tested on 2100 bioanalyzer using a capillary electrophoresis kit, High Sensitivity DNA Kit (Agilent, Cat. No. 5067-4626). The fragment analysis results are shown in FIG. 11, FIG. 12 and FIG. 13.

On the basis of one-step assembly, the PCA system can be reduced to 2-5 μL, and the components of PCR reaction system can be directly added into the PCA reaction tube for one-tube assembly.

(4) Clone Sequencing

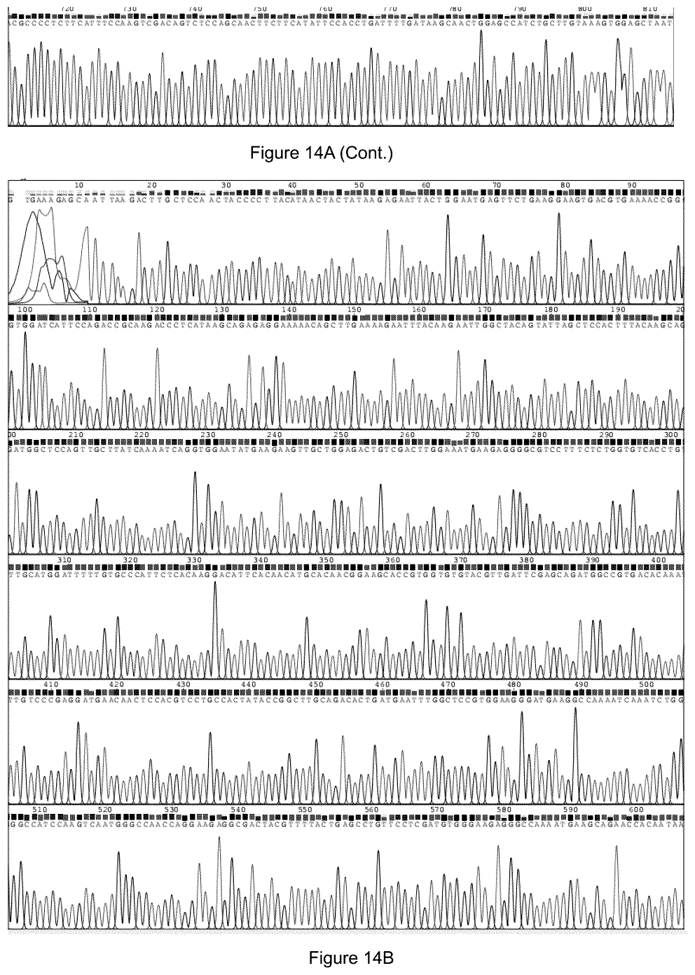

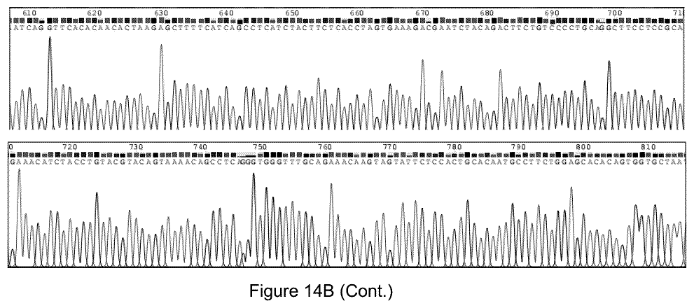

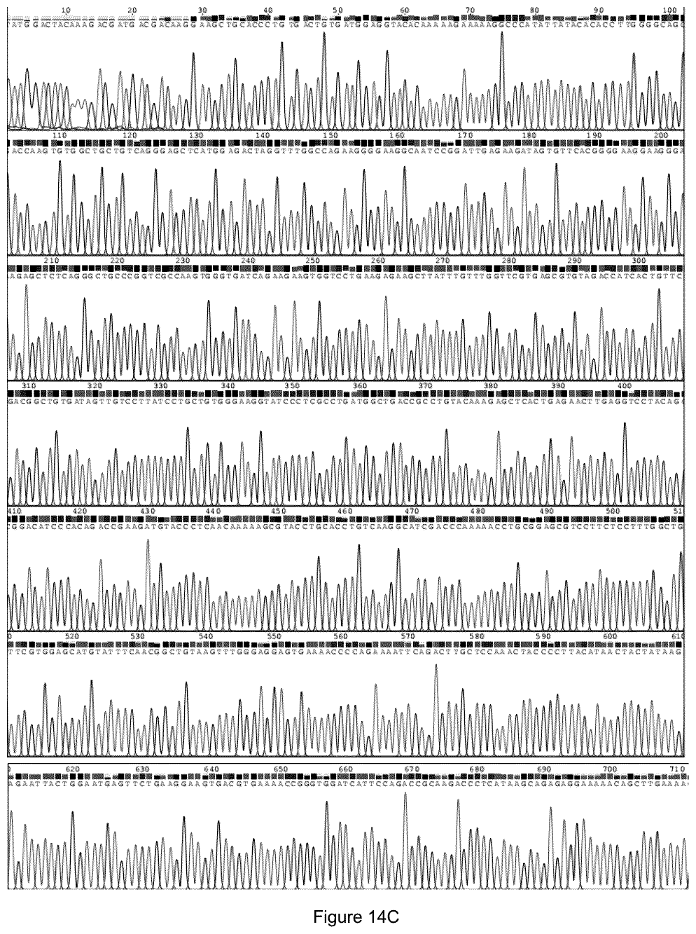

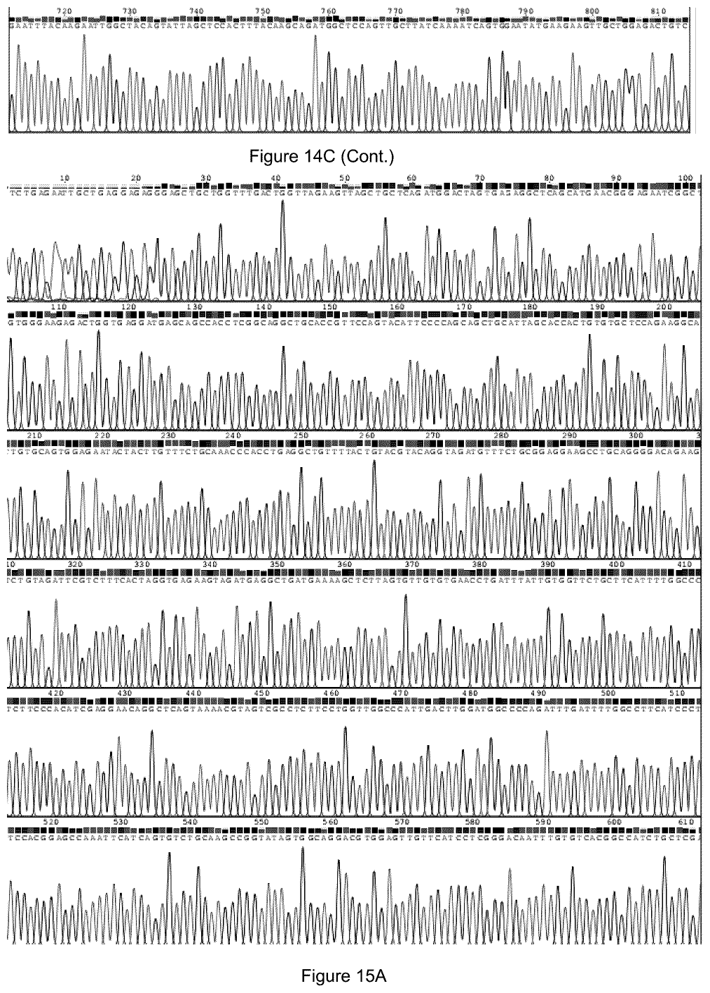

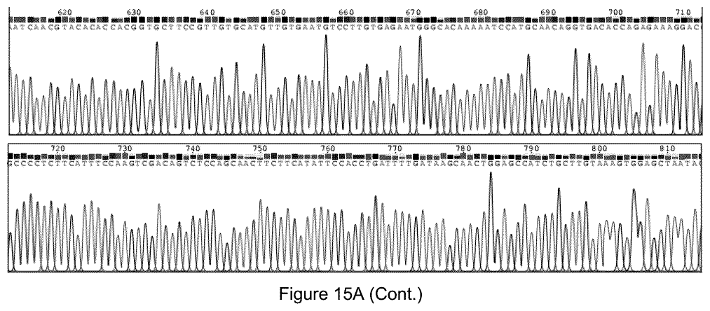

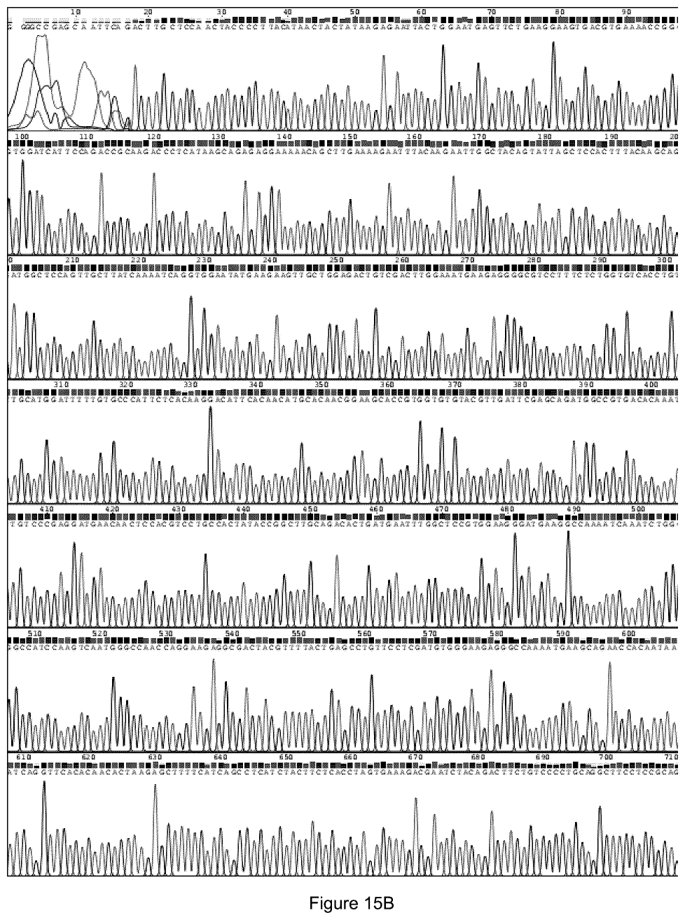

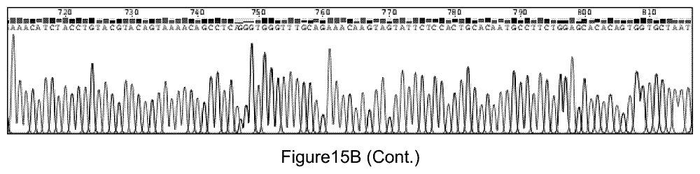

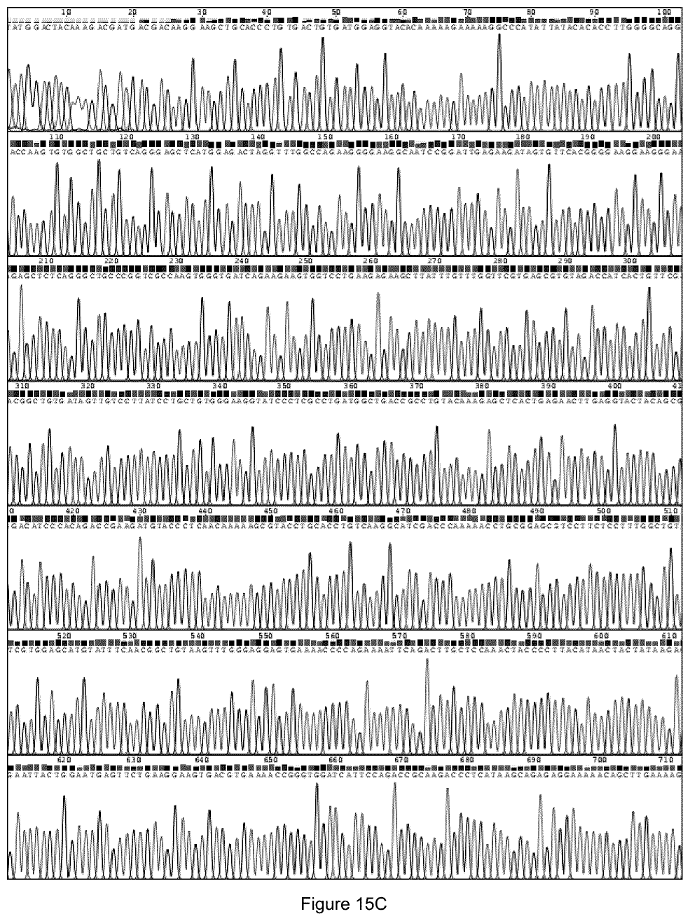

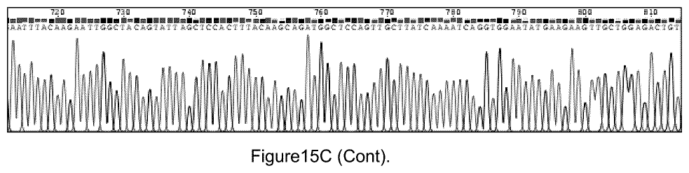

After ligating the PCR product obtained in step (3) and the error-corrected product with the T vector, the plasmids were transferred into Escherichia coli DH5α competent cells, and every 10 to 16 positive clones were picked for first-generation sequencing. The sequencer used was ABI 3730 XL, and it was found that all the sequence results showed that the fragments of the target length had been successfully synthesized, and it was ensured that at least one strain contained completely correct sequences, while other sequences contained from 1 to 2 mutation sites. The sequencing results are shown in FIGS. 14-19. Among them, FIG. 14 is the sequencing result of strain 1 (including FIG. 14-A, FIG. 14-B and FIG. 14-C, three Sanger sequencing fragments were fully sequenced), FIG. 15 is the sequencing result of strain 2 (including FIG. 15-A, FIG. 15-B and FIG. 15-C, three Sanger sequencing fragments were fully sequenced), the sequencing result of strain 1 was correct, and the sequencing result of strain 2 had 1 base error. The above results were detected by Agilent 2100 Bioanalyzer, showing broad tailed peaks of uncorrected genes, and sequencing shows an error rate of about 1/500 to 1/1000.

Then two rounds of error correction were performed using CorrectASE. After the first and second rounds of correction, on the 2100 bioanalyzer, a sharper peak was detected, indicating a lower error rate. From the error-corrected products obtained in step (3), 2 to 4 colonies were picked for sequencing to obtain completely correct gene clones. Sequencing results showed that after error correction, the sequencing showed an error rate of about 1/3000-1/10000.

### INDUSTRIAL APPLICATION

The high-throughput automated gene synthesis system based on cluster arrays completes the high-throughput oligonucleotide synthesis through the funnel-shaped pores in cluster structure. Then these cluster arrays are one-to-one automatically recovered into the wells of standard SBS plates to form oligonucleotide pools for subsequent gene assembly. The yield of oligonucleotides reaches picomole level, which can just meet the needs of gene splicing without amplification.

Compared with the traditional gene synthesis method, it avoids both a large number of the manual operations of mixing oligonucleotides, and waste caused by the nanomole level products of traditional oligonucleotide synthesis approach. Compared with oligonucleotides synthesized based on microarray chips, the yield of a single oligonucleotide is higher which can be directly used for subsequent gene assembly without amplification. There is no need for a PCR splitting step in high-throughput oligonucleotide sub-pools. At the same time, errors caused by amplification can be effectively reduced, thereby reducing the error rate.

At the same time, ultra-long oligonucleotides can realize one-step splicing and simplify the operation steps. The synthesis amount of each oligonucleotide just meets the picomole level of gene splicing, which reduces the synthesis cost. At the same time, the cluster synthesis of unique oligonucleotides is innovatively connected with the standard microwell plate for downstream gene splicing, which achieves a higher automation level than traditional multi-step splicing.

The present invention solves the current bottlenecks in the field of gene synthesis, such as low-throughput, and cumbersome manual operation, provides a commercialized and low-cost high-throughput automated gene synthesis method.

1-14. (canceled)

15. A high-throughput gene synthesis device based on cluster arrays, including a substrate and a microwell plate;
the substrate is provided with a plurality of clusters of micropores; the inner wall surface of the micropore is chemically modified as a solid phase carrier for nucleic acid synthesis, or the micropore is filled with solid phase carriers for nucleic acid synthesis;
a plurality of clusters of the micropores are arranged in a cluster array, and each cluster of the micropores has the same size and corresponding position as each well on the microwell plate.

16. The gene synthesis device according to claim 15, wherein the micropore is a funnel-shaped micropore or a cylindrical micropore;
the opening of the funnel-shaped micropore is a large opening end.

17. The gene synthesis device according to claim 16, wherein the substrate is a silicon wafer.

18. The gene synthesis device according to claim 17, wherein the micropore is prepared by the MEMS micro-nano processing method.

19. The gene synthesis device according to claim 16, wherein the substrate is a polymer plastic plate.

20. The gene synthesis device according to claim 16, wherein the micropore is prepared by 3D printing or injection molding.

21. The gene synthesis device according to claim 15, wherein the solid phase carriers are glass microspheres or polystyrene microspheres.

22. The gene synthesis device according to claim 15, wherein the solid phase carriers are immobilized in the micropore as follows:
mixing the solid phase carriers with high-density polyethylene spheres, and sintering.

23. The gene synthesis device according to claim 15, wherein each cluster of the micropores includes from 4 to 68 of the micropores.

24. The gene synthesis device according to claim 15, wherein the microwell plate is a standard SBS plate.

25. Any of the following methods:
(i) a method for oligonucleotide synthesis;
(ii) a method for nucleic acid synthesis;
(iii) a method for synthesizing oligonucleotides and genes.

26. The method according to claim 25, wherein the method for oligonucleotide synthesis comprises the steps of:
(1) Phosphoramidite monomers or auxiliary reagents are added to the micropores of the gene synthesis device utilizing a liquid dispensing device;
(2) The reaction is conducted on the solid phase carriers within the micropores to synthesize the oligonucleotides;
(3) The gene synthesis device is then matched with the microwell plate;
(4) The oligonucleotides synthesized in each cluster of the micropores are recovered into a single well within the microwell plate.

27. The method according to claim 26, wherein the liquid dispensing device is a micro-nano litre level liquid dispensing head.

28. The method according to claim 25, wherein the method for nucleic acid synthesis comprises the steps of:
(1) Synthesis of oligonucleotides within the micropores of a gene synthesis device, using any of the methods specified in claim 25;
(2) Recovery of the oligonucleotides from all the micropores in a cluster into a single well of a microwell plate;
(3) Direct splicing of the recovered oligonucleotides to obtain the synthesized nucleic acid.

29. The method according to claim 25, wherein the method for synthesizing oligonucleotides and genes comprises the use of a gene synthesis device, wherein the gene synthesis device includes a substrate and a microwell plate;
the substrate is provided with a plurality of clusters of micropores; the inner wall surface of the micropore is chemically modified as a solid phase carrier for nucleic acid synthesis, or the micropore is filled with solid phase carriers for nucleic acid synthesis; and
a plurality of clusters of the micropores are arranged in a cluster array, and each cluster of the micropores has the same size and corresponding position as each well on the microwell plate.
